(19) United States
(12) Patent Application Publication
(10) Pub. No.: US 2007/0161566 A1
(43) Pub. Date: Jul. 12, 2007

(54) Method of treating multiple sclerosis

(75) Inventor: Irit Pinchasi, Raanana (IL)

Correspondence Address: COOPER & DUNHAM, LLP, 1185 AVENUE OF THE AMERICAS, NEW YORK, NY, 10036, US

(73) Assignee: Teva Pharmaceutical Industries, Ltd.

(21) Appl. No.: 11/651,212

(22) Filed: Jan. 9, 2007

Related U.S. Application Data
(60) Provisional application No. 60/758,580, filed on Jan. 11, 2006.

(51) Int. Cl. A61K 38/16

(52) U.S. Cl. 514/12

(57) ABSTRACT

This invention provides a method of alleviating a symptom of a patient suffering from a relapsing form of multiple sclerosis which comprises periodically administering to the patient by subcutaneous injection a single dose of a pharmaceutical composition comprising 40 mg of glatiramer acetate so as to thereby alleviate the symptom of the patient. This invention also provides a method of reducing Gd-enhancing lesions in the brain and a pharmaceutical composition in a unit dosage.

This application claims benefit of U.S. Provisional Application No. 60/758,580, filed Jan. 11, 2006 the contents of which are hereby incorporated by reference.

Throughout this application various publications are referenced by their full citations. The disclosures of these publications in their entireties are hereby incorporated by reference into this application in order to more fully describe the state of the art to which this invention pertains.

### BACKGROUND OF THE INVENTION

One of the more common chronic neurologic diseases in human adults is multiple sclerosis (“MS”). MS is a chronic, inflammatory CNS disease characterized pathologically by demyelination. MS has also been classified as an autoimmune disease. MS disease activity can be monitored by magnetic resonance imaging (MRI) of the brain, accumulation of disability, as well as rate and severity of relapses.

There are five main forms of multiple sclerosis: 1) benign multiple sclerosis; 2) relapsing-remitting multiple sclerosis (RRMS); 3) secondary progressive multiple sclerosis (SPMS); 4) primary progressive multiple sclerosis (PPMS); and 5) progressive-relapsing multiple sclerosis (PRMS) (What are the Types of Multiple Sclerosis?, 2005 <http://imaginis.com/multiple-sclerosis/types-of-ms.asp? mode=1>). Chronic progressive multiple sclerosis is a term used to collectively refer to SPMS, PPMS, and PRMS (Types of Multiple Sclerosis (MS), 2005 <http://www.themcfox.com/multiple-sclerosis/types-of-ms/types-of-multiple-sclerosis.htm>). The relapsing forms of multiple sclerosis are SPMS with superimposed relapses, RRMS and PRMS.

Benign multiple sclerosis is a retrospective diagnosis which is characterized by 1-2 exacerbations with complete recovery, no lasting disability and no disease progression for 10-15 years after the initial onset. Benign multiple sclerosis may, however, progress into other forms of multiple sclerosis. Patients suffering from RRMS experience sporadic exacerbations or relapses, as well as periods of remission. Lesions and evidence of axonal loss may or may not be visible on MRI for patients with RRMS. SPMS may evolve from RRMS. Patients afflicted with SPMS have relapses, a diminishing degree of recovery during remissions, less frequent remissions and more pronounced neurological deficits than RRMS patients. Enlarged ventricles, which are markers for atrophy of the corpus callosum, midline center and spinal cord, are visible on MRI of patients with SPMS. PPMS is characterized by a steady progression of increasing neurological deficits without distinct attacks or remissions. Cerebral lesions, diffuse spinal cord damage and evidence of axonal loss are evident on the MRI of patients with PPMS. PRMS has periods of acute exacerbations while proceeding along a course of increasing neurological deficits without remissions. Lesions are evident on MRI of patients suffering from PRMS (Multiple sclerosis: its diagnosis, symptoms, types and stages, 2003 <http://www.albany.net/˜tjc/multiple-sclerosis.html>). Glatiramer acetate (GA), a mixture of polypeptides which do not all have the same amino acid sequence, is marketed under the tradename Copaxone®. GA comprises the acetate salts of polypeptides containing L-glutamic acid, L-alanine, L-tyrosine and L-lysine at average molar fractions of 0.141, 0.427, 0.095 and 0.338, respectively. The average molecular weight of Copaxone® is between 5,000 and 9,000 daltons. (“Copaxone”, Physician's Desk Reference, (2005), Medical Economics Co., Inc., (Montvale, N.J.), 3115.) Chemically, glatiramer acetate is designated L-glutamic acid polymer with L-alanine, L-lysine, L-tyrosine, acetate (salt). Its structural formula is:
(Glu, Ala, Lys, Tyr)ₓ●χCH₃COOH (C₅H₉NO₄●C₃H₇NO₂●C₆H₁₄N₂O₂ ●C₉H₁₁NO₃)ₓ●χC₂H₄O₂CAS-147245-92-9
(“Copaxone”, Physician's Desk Reference, (2000), Medical Economics Co., Inc., (Montvale, N.J.), 3115.) Copaxone® (20 mg glatiramer acetate injection) is an approved therapy for patients with relapsing remitting multiple sclerosis (RRMS).

Glatiramer acetate has also been disclosed for use in the treatment of other autoimmune diseases (U.S. Patent Publication No. 2002/0055466 A1 (R. Aharoni et al.), inflammatory non-autoimmune diseases (U.S. Patent Publication No. 2005/0014694 A1 (V. Wee Yong et al.); and U.S. Patent Application No. 2002/0077278 A1, published Jun. 20, 2002 (Young et al.)) and other diseases (U.S. Patent Publication Nos. 2003/0004099 A1 and 2002/0037848 A1 (Eisenbach-Schwartz, et al.); U.S. Pat. No. 6,514,938 B1, issued Feb. 4, 2003 (Gad et al.); PCT International Publication No. WO 01/60392, published Aug. 23, 2001 (Gilbert et al.); PCT International Publication No. WO 00/27417, published May 19, 2000 (Aharoni et al.); and PCT International Publication No. WO 01/97846, published Dec. 27, 2001 (Moses et al.)).

The 20 mg/day subcutaneous dose has been shown to reduce the total number of enhancing lesions in MS patients as measured by MRI (G. Comi et al., European/Canadian Multicenter, Double-Blind, Randomized, Placebo-Controlled Study of the Effects of Glatiramer Acetere on Magnetic Resonance Imaging-Measured Disease Activity and Burden in Patients with Relapsing Multiple Sclerosis, Ann. Neurol. 49:290-297 (2001)). However, disclosed herein is the finding that administration of glatiramer acetate at a dose of 40 mg/day significantly improves efficacy but does not have a corresponding increase in adverse reactions experienced by the patient.

### SUMMARY OF THE INVENTION

This invention provides a method of alleviating a symptom of a patient suffering from a relapsing form of multiple sclerosis which comprises periodically administering to the patient by subcutaneous injection a single dose of a pharmaceutical composition comprising 40 mg of glatiramer acetate so as to thereby alleviate the symptom of the patient.

This invention also provides a method of reducing MRI-monitored disease activity and burden of a patient suffering from multiple sclerosis which comprises periodically administering to the patient by subcutaneous injection a single dose of a pharmaceutical composition comprising 40 mg of glatiramer acetate.

This invention further provides a pharmaceutical composition in a unit dosage injectable form comprising 40 mg of glatiramer acetate and a pharmaceutically acceptable carrier.

This invention also provides a use of glatiramer acetate in the manufacture of a pharmaceutical composition comprising a 40 mg glatiramer acetate for subcutaneous administration to alleviate a symptom of a relapsing form of multiple sclerosis in a human patient.

### BRIEF DESCRIPTION OF THE FIGURES

FIG. 1. Mean±SE Of T1 Gd-Enhancing Lesions by Month−Mean±SE of T1 Gd-enhancing lesions by month comparing 20 mg and 40 mg per day GA dosages.

FIG. 2. Primary Analysis: ITT Cohort (N=81) Cumulative Number of Tl-Enhancing Lesions at Months 7, 8 and 9—primary analysis of the cumulative number of T1 Gd-enhanced lesions at months 7, 8, and 9 comparing 20 mg and 40 mg per day GA dosages for the ITT cohort (n=81).

FIG. 3. Post Hoc Analysis: ITT (N=84) Cumulative Number of T1 GD-Enhancing Lesions at Month 3—Post-hoc analysis of the cumulative number of T1-enhancing lesions at month 3 for ITT (n=84) comparing 20 mg and 40 mg per day GA dosages.

FIG. 4. Number of Confirmed Relapses on Trial-Graphic comparison of the number of confirmed relapse in the trial between the 20 mg GA per day and 40 mg GA per day dosage groups.

FIG. 5. Time to First Confirmed Relapse-Graphic comparison of the time to first confirmed relapse between the 20 GA mg per day and 40 mg GA per day dosage groups.

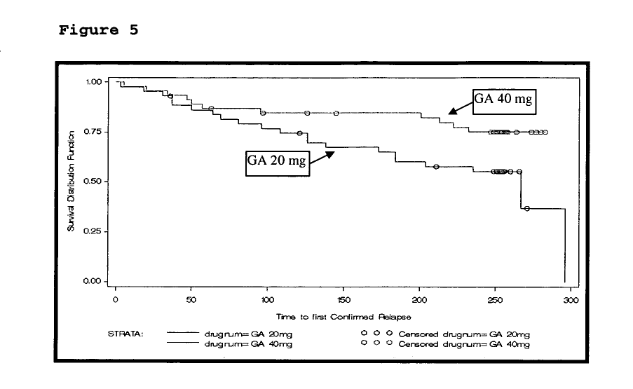

FIG. 6. Mean±SE of New T2 Lesions by Month—A graphic comparison of the mean±SE new lesions by month between the 20 mg GA per day and the 40 mg GA per day dosage groups.

FIG. 7. Cumulative Number of New T2 Gd-Enhancing Lesions at Months 8 and 9 (N=81)—a graphic comparison of the adjusted means of the cumulative number of new T2 Gd-enhancing lesions at months 8 and 9 between the 20 mg GA per day and 40 mg GA per day dosage groups.

### DETAILED DESCRIPTION OF THE INVENTION

This invention provides a method of alleviating a symptom of a patient suffering from a relapsing form of multiple sclerosis which comprises periodically administering to the patient by subcutaneous injection a single dose of a pharmaceutical composition comprising 40 mg of glatiramer acetate so as to thereby alleviate the symptom of the patient.

In an embodiment, the periodic administration is daily.

In another embodiment, the periodic administration is every other day.

In yet another embodiment, the relapsing form of multiple sclerosis is relapsing-remitting multiple sclerosis.

In a further embodiment, the symptom is the frequency of relapses.

In an embodiment, the pharmaceutical composition is in the form of a sterile solution.

In another embodiment, the pharmaceutical composition further comprises mannitol.

In yet another embodiment, the pharmaceutical composition has a pH in the range of 5.5 to 8.5. In an embodiment, the pharmaceutical composition has a pH in the range of 5.5 to 7.0.

In a further embodiment, the pharmaceutical composition is in a prefilled syringe and is self-administered by the patient.

This invention also provides a method of reducing MRI-monitored disease activity and burden of a patient suffering from multiple sclerosis which comprises periodically administering to the patient by subcutaneous injection a single dose of a pharmaceutical composition comprising 40 mg of glatiramer acetate.

In an embodiment, reducing MRI-monitored disease activity and burden is reducing the mean cumulative number of Gd-enhancing lesions in the brain of the patient.

In another embodiment, reducing MRI-monitored disease activity and burden is reducing the mean number of new T2 lesions in the brain of the patient.

In any of the above embodiments of the method, the periodic administration to the patient of the single dose of a pharmaceutical composition comprising 40 mg of glatiramer acetate further reduces a symptom of MS. In an embodiment, the symptom may be the frequency of relapses.

In any embodiment of a method of reducing MRI-monitored disease activity and burden, the periodic administration is daily. The periodic administration may alternatively be every other day.

In further embodiments of the method, the patient is suffering from a relapsing form of multiple sclerosis. In an embodiment, the relapsing form of multiple sclerosis is relapsing-remitting multiple sclerosis.

In further embodiments of the method, the pharmaceutical composition is in the form of a sterile solution.

In yet further embodiments of the method, the pharmaceutical composition further comprises mannitol.

In further embodiments of the method, the pharmaceutical composition has a pH in the range of 5.5 to 8.5. In an embodiment, the pharmaceutical composition may have a pH in the range of 5.5 to 7.0.

In a further embodiment of the method, the pharmaceutical composition is in a prefilled syringe and is self-administered by the patient.

This invention further provides a pharmaceutical composition in a unit dosage injectable form comprising 40 mg of glatiramer acetate and a pharmaceutically acceptable carrier.

In an embodiment, the pharmaceutical composition is in the form of a sterile solution.

In another embodiment, the pharmaceutically acceptable carrier is mannitol.

In yet another embodiment, the pharmaceutical composition has a pH in the range of 5.5 to 8.5. In an embodiment, the pharmaceutical composition may have a pH in the range of 5.5 to 7.0.

In a further embodiment, the pharmaceutical compositions is in a prefilled syringe.

This invention also provides a use of glatiramer acetate in the manufacture of a pharmaceutical composition comprising a 40 mg glatiramer acetate for subcutaneous administration to alleviate a symptom of a relapsing form of multiple sclerosis in a human patient.

In an embodiment of the use, the relapsing form of multiple sclerosis is relapsing-remitting multiple sclerosis.

In another embodiment of the use, the symptom is the frequency of relapses.

In a further embodiment of the use, the pharmaceutical composition is in the form of a sterile solution for once daily administration.

In an embodiment of the use, the pharmaceutical composition further comprises mannitol.

In another embodiment of the use, the pharmaceutical composition is in the form of a sterile solution having a pH in the range 5.5 to 8.5. In an embodiment, the pharmaceutical composition is the in the form of a sterile solution having a pH in the range 5.5 to 7.0.

In yet another embodiment of the use, the pharmaceutical composition is in a prefilled syringe.

Definitions

As used herein, immediate post injection reaction (IRPR) refers to a reaction such as, palpitations, feeling hot, flushing, hot flushes, tachycardia, dyspnoea, chest discomfort, chest pain, and non-cardiac chest pain that occurs immediately following injection. Reactions may also include asthenia, back pain, bacterial infection, chills, cyst, face edema, fever, flu syndrome, infection, injection site erythema, injection site hemorrhage, injection site induration, injection site inflammation, injection site mass, injection site pain, injection site pruritus, injection site urticaria, injection site welt, neck pain, pain, migrane, syncope, tachycardia, vasodilatation, anorexia, diarrhea, gastroenteritis, gastrointestinal disorder, nausea, vomiting, ecchymosis, peripheral edema, arthralgia, agitation, anxiety, confusion, foot drop, hypertonia, nervousness, nystagmus, speech disorder, tremor, vertigo, bronchitis, dyspnea, laryngismus, rhinitis, erythema, herpes simplex, pruritus, rash, skin nodule, sweating, urticaria, ear pain, eye disorder, dysmenorrheal, urinary urgency, and vaginal moniliasis.

As used herein, injection site reaction (ISR) refers to a reaction such as erythema, hemorrhage, induration, inflammation, mass, pain, pruritus, urticaria, and welt that occurs immediately around the site of injection.

As used herein, the term Gd-enhancing lesions refers to lesions that result from a breakdown of the blood-brain barrier, which appear in contrast studies using gandolinium contrast agents. Gandolinium enhancement provides information as to the age of a lesion, as Gd-enhancing lesions typically occur within a six week period of lesion formation.

As used herein, the term T1-weighted MRI images refers to an MR-image that emphasizes T1 contrast by which lesions may be visualized. Abnormal areas in a T1-weigted MRI image are “hypointense” and appear as dark spots. These spots are generally older lesions.

As used herein, the term “unit dosage” refers to physically discrete units suited as single administration dose for a subject to be treated, containing a therapeutically effective quantity of active compound in association with the required pharmaceutical carrier, e.g., a syringe.

This invention is illustrated in the Examples section which follows. This section is set forth to aid in an understanding of the invention but is not intended to, and should not be construed to, limit in any way the invention as set forth in the claims which follow thereafter.

### EXAMPLES

### Example 1

### 9 Month 40 mg Glatiramer Acetate Treatment

Objectives:

To evaluate the safety and efficacy of 40 mg of glatiramer acetate treatment for 9 months, compared to Copaxone® (20 mg formulation) both administered by daily subcutaneous injection, as reflected primarily by Gd-enhancing lesions on T1-weighted MRI images, and by relapse rate.

Preparation of 40 mg GA Injection:

Quantitative Composition of Copaxone 40 mg/PFS Injection

| Name of Ingredient | Unit Dose, mg/mL | |
|---|---|---|
| Glatiramer Acetate DS | 40 | mg |
| Mannitol | 40 | mg |
| Sterilized Water for | 1.0 | mL |
| Injection | | |

Copaxone (Glatiramer Acetate Injection) 40 mg/PFS is a solution containing dose of 40 mg of the drug substance and 40 mg of Mannitol USP in 1 mL sterilized water for injection. Compounding procedures including dissolving of Glatiramer Acetate drug substance (DS) (providing a final concentration of 40 mg/mL of anhydrous form) in water for injection with addition of 40 mg/mL Mannitol. The DS is the active substance only. The drug product (DP) is the mixture of carrier including the active substance.

Design/Methods:

Ninety (90) eligible subjects with at least one Gd-enhancing lesion at screening (month −1) were randomized into 9-month, double-blind, parallel group study and received sc injection of either 40 mg/d or 20 mg/d GA. Subjects underwent MRI scans at months 3, 7, 8 and 9. Neurological examinations were performed in study centers at screening, baseline (month 0), and at months 3, 6, and 9. Suspected relapses were confirmed by the examining neurologist within 7 days.

The primary efficacy endpoint was the total number of Gd-enhancing lesions on T1-weighted images, as measured at months 7, 8 and 9. The difference between the two treatment arms was assessed using a Poisson regression model accounting for study-site, and baseline Gd-enhancing lesion counts.

Results:

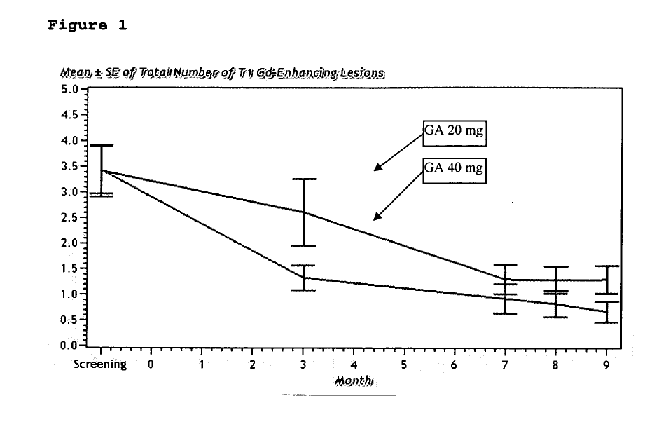

In 90 RRMS patients, age ranges between 23.4-51.2 years (mean±SE 37.2±0.7). At entry, mean duration of disease was 3.5±0.5 years (range: 0-17.5 years), mean EDSS 2.0±0.1 (range: 0-4.5), and mean annual relapse rate (ARR) based on patients' entire history was 1.5±0.1 (range: 1-5). Mean Gd-enhancing lesions at screening was 3.4±0.34 (n=89). (See FIG. 1) The two groups were comparable in their MS demographic, clinical and MRI parameters at entry.

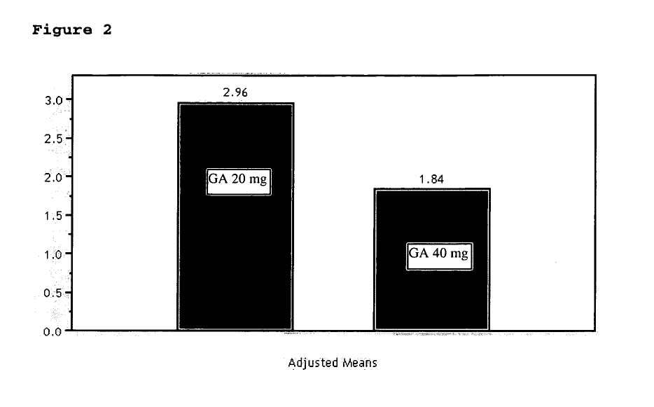

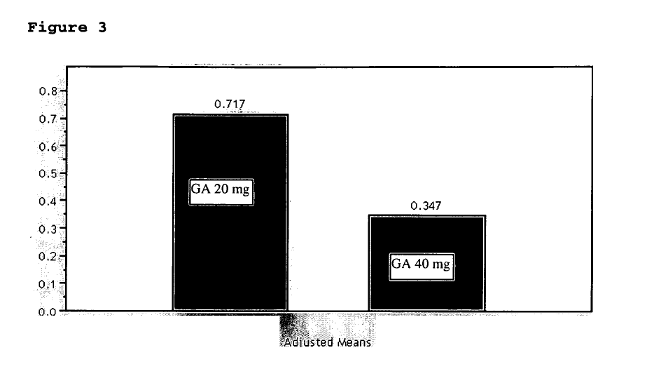

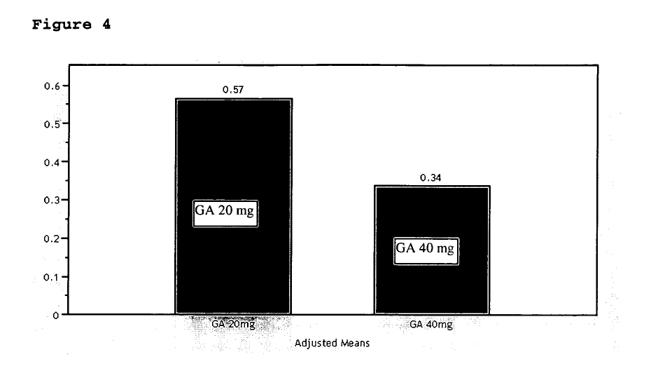

A 38% greater reduction (RR=0.62, 95% CI 0.36-1.08, p=0.0898) in favor of 40 mg vs. 20 mg in the mean cumulative number of Gd-enhancing lesions at month 7, 8 and 9 (mean±SD 0.79±1.36 vs. 1.32±1.51 lesions per scan for the 40 and 20 mg groups, respectively) (See FIG. 2 and Table 1)—was observed. This difference has emerged as early as 3 months (1.33±1.58 lesions vs. 2.61±4.22 lesions for the 40 and 20 mg groups, respectively, p=0.005). (See FIG. 3 and Table 2). The significance of the result at 3 months is shown in FIG. 3. When compared to baseline, the risk of having enhancement at month 7, 8 and 9 was reduced by 75% (RR 0.25, 95% CI 0.15-0.40, p<0.0001) in the 40 mg/day and by 65% (RR 0.35, 95% CI 0.24-0.53, p<0.0001) in the 20 mg/day group. Mean relapse rate after 9 months reached 0.57 and 0.34 for the 20 mg and 40 mg groups respectively, with a delay in time of 20% patients their first on trial relapse of 213 and 80 days for the 40 and 20 mg/day groups, respectively. (See FIG. 4 and Table 3) The safety profile of the 40 mg/d dose is essentially similar to the currently available 20 mg/day dose with a slight tendency of higher incidence of IPIR.

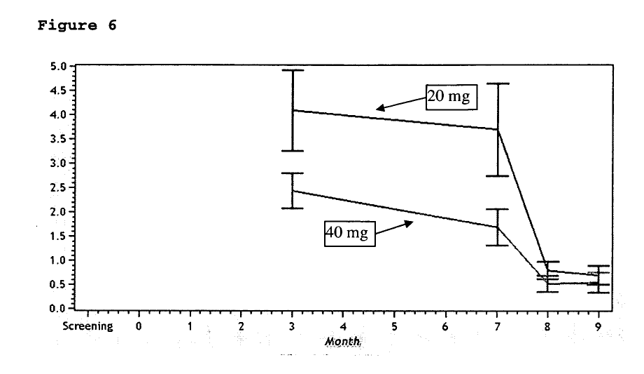

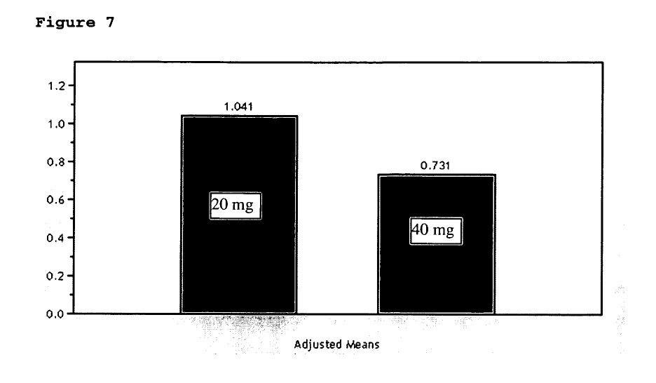

FIG. 6 shows the mean±SE of new T2 lesions by month, from month 3 to month 9, of the 20 mg GA per day and 40 mg GA per day dosage groups. FIG. 7 and Table 6 show the cumulative number of new T2 Gd-enhancing lesions at months 8 and 9 in the 20 mg GA per day and 40 mg GA per day dosage groups.

TABLE 1
Primary Analysis: ITT Cohort (N = 81) Cumulative
Number of T1 Gd-Enhancing Lesions at Months 7, 8, and 9

| | |
|---|---|
| GA 20 mg-Adjusted Means [95% CL] | 2.96 - [1.90, 4.59] |
| GA 40 mg-Adjusted Means [95% CL] | 1.84 - [1.11, 3.05] |
| RR (Relative Risk) [95% CL] | 0.62 - [0.36, 1.08] |
| LR-Test-P value | 0.0898 |

(See also FIG. 2)

TABLE 2
Post Hoc - Analysis: ITT (n = 84) Cumulative Number
of T1 Gd-Enhancing Lesions at Month 3

| | |
|---|---|
| GA 20 mg-Adjusted Means [95% CL] | 0.72 - [0.47, 1.10] |
| GA 40 mg-Adjusted Means [95% CL] | 0.35 - [0.21, 0.58] |
| RR (Relative Risk) [95% CL] | 0.48 - [0.29, 0.82] |
| LR-Test-P value | 0.0051 |

(See also FIG. 3)

TABLE 3
Number of Confirmed Relapses on Trial

| | |
|---|---|
| GA 20 mg-Adjusted Means [95% CL] | 0.57 - [0.37, 0.86] |
| GA 40 mg-Adjusted Means [95% CL] | 0.34 - [0.20, 0.57] |
| RR (Relative Risk) [95% CL] | 0.59 - [0.31, 1.16] |
| LR-Test-P value | 0.1202 |

(See also FIG. 4)

TABLE 4
Potential IPIR: Immediate Post Injection Reaction

| | | | | GA 40 mg (N = 46) | | | |
|---|---|---|---|---|---|---|---|
| | GA 20 mg (N = 44) | | | | | % of | |
| | No. of | No. of | % of | No. of | No. of | Sub- | Δ |
| | Reports | Subjects | Subjects | Reports | Subjects | jects | (%) |
| Any | 39 | 10 | 22.7 | 52 | 15 | 32.6 | 9.9 |
| symptom | | | | | | | |
| Palpi- | 1 | 1 | 2.3 | 10 | 5 | 10.9 | 8.6 |
| tations | | | | | | | |
| Feeling | 13 | 6 | 13.6 | 16 | 9 | 19.5 | 5.9 |
| Hot/ | | | | | | | |
| Flushing/ | | | | | | | |
| Hot Flush | | | | | | | |
| Tachy- | — | — | — | 3 | 2 | 4.3 | 4.3 |
| cardia | | | | | | | |
| Dyspnoea | 10 | 6 | 13.6 | 17 | 7 | 15.2 | 1.6 |
| Chest | 15 | 6 | 13.6 | 6 | 4 | 8.7 | −4.9 |
| Dis- | | | | | | | |
| comfort/ | | | | | | | |
| Chest | | | | | | | |
| Pain/ | | | | | | | |
| Non- | | | | | | | |
| Cardiac | | | | | | | |
| Chest Pain | | | | | | | |

TABLE 5
Injection Site Reactions

| GA 20 mg (N = 44) | | | GA 40 mg (N = 46) | | |
|---|---|---|---|---|---|
| No. of | No. of | % of | No. of | No. of | % of |
| Reports | Subjects | Subjects | Reports | Subjects | Subjects |
| 113 | 38 | 86.4 | 126 | 39 | 84.8 |

No Injection Site Necrosis or Lipoatrophy

TABLE 6
Cumulative Number of New T2 Gd-Enhancing Lesions at Months 8 and 9
(N = 81)

| | |
|---|---|
| GA 20 mg-Adjusted Means [95% CL] | 1.04 - [0.58, 1.86] |
| GA 40 mg-Adjusted Means [95% CL] | 0.73 - [0.40, 1.35] |
| RR (Relative Risk) [95% CL] | 0.70 - [0.38, 1.30] |
| LR-Test-P value | 0.2562 |

(See also FIG. 7)

Conclusions:

The increased efficacy observed with 40 mg/day GA in reducing MRI-measured disease activity and relapse rate indicates that it is well tolerated and can improve the treatment of RRMS patients. The improvement in efficacy, however, is not accompanied by a corresponding increase of adverse reactions which would be expected upon a doubling of the administered dose.

Also observed was the accelerated rate at which the 40 mg/day dose became effective as compared to the 20 mg/day dose. This was unexpected. Specifically, the 40 mg/day dose showed efficacy, as measured by MRI, by the third month, whereas the 20 mg/day dose did not show efficacy until the sixth month. The results at three months comparing the 40 mg/day dosage with the 20 mg/day dosage are shown in FIG. 3 and Table 2 above.

The increased efficacy observed with a 40 mg/day GA administration was also unexpected in view of another finding that the administration of 15 mg twice per day (30 mg per day) of GA did not produce statistically significant difference between the placebo and treated groups for the arrest or reversal of disease progress in patients affected by the chronic-progressive MS (Bornstein M. B. et al., A placebo-controlled, double-blinded, randomized, two-center, pilot trial of Cop 1 in chronic progressive multiple sclerosis, Neurology 41:533-539 (1991)). In this previous double-blinded, randomized, placebo-controlled trial of GA in chronic progressive MS patients, patients received 15 mg GA twice daily. Patients continued in the study until they had demonstrated either a confirmed worsening over their baseline EDSS score maintained for at least 3 months or had completed 24 months of treatment. There was no statistically significant difference between the results of placebo groups from the results of patients that received 30 mg per day.

1. A method of alleviating a symptom of a patient suffering from a relapsing form of multiple sclerosis which comprises periodically administering to the patient by subcutaneous injection a single dose of a pharmaceutical composition comprising 40 mg of glatiramer acetate so as to thereby alleviate the symptom of the patient.

2. The method of claim 1, wherein the periodic administration is daily.

3. The method of claim 1, wherein the periodic administration is every other day.

4. The method of claim 1, wherein the relapsing form of multiple sclerosis is relapsing-remitting multiple sclerosis.

5. The method of claim 1, wherein the symptom is the frequency of relapses.

6. The method of claim 1, wherein the pharmaceutical composition is in the form of a sterile solution.

7. The method of claim 1, wherein the pharmaceutical composition further comprises mannitol.

8. The method of claim 1, wherein the pharmaceutical composition has a pH in the range of 5.5 to 8.5.

9. The method of claim 8, wherein the pharmaceutical composition has a pH in the range of 5.5 to 7.0.

10. The method of claim 1, wherein the pharmaceutical composition is in a prefilled syringe and is self-administered by the patient.

11. A method of reducing MRI-monitored disease activity and burden of a patient suffering from multiple sclerosis which comprises periodically administering to the patient by subcutaneous injection a single dose of a pharmaceutical composition comprising 40 mg of glatiramer acetate.

12. The method of claim 11, wherein reducing MRI-monitored disease activity and burden is reducing the mean cumulative number of Gd-enhancing lesions in the brain of the patient.

13. The method of claim 11, wherein reducing MRI-monitored disease activity and burden is reducing the mean number of new T2 lesions in the brain of the patient.

14. The method of claim 11, wherein the periodic administration to the patient of the single dose of a pharmaceutical composition comprising 40 mg of glatiramer acetate further reduces a symptom of MS.

15. The method of claim 14, wherein the symptom is the frequency of relapses.

16. A pharmaceutical composition in a unit dosage injectable form comprising 40 mg of glatiramer acetate and a pharmaceutically acceptable carrier.

17. The pharmaceutical composition of claim 16, wherein the pharmaceutical composition is in the form of a sterile solution.

18. The pharmaceutical composition of claim 16, wherein the pharmaceutically acceptable carrier is mannitol.

19. The pharmaceutical composition of claim 16 having a pH in the range of 5.5 to 8.5.

20. The pharmaceutical composition of claim 19 having a pH in the range of 5.5 to 7.0.

21. The pharmaceutical composition of claim 16 in a prefilled syringe.

22-29. (canceled)
